(19) United States
(12) Patent Application Publication
(10) Pub. No.: US 2005/0080373 A1
(43) Pub. Date: Apr. 14, 2005

(54) Apparatus and a method for treating blood related illnesses

(76) Inventor: Xiaoling Wang, Princeton Junction, NJ (US)

Correspondence Address: Mr. Walter J. Tencza, Jr., 10 Station Place, Suite 3, Metuchen, NJ, 08840, US

(21) Appl. No.: 10/941,614

(22) Filed: Sep. 15, 2004

Related U.S. Application Data
(60) Provisional application No. 60/509,586, filed on Oct. 9, 2003.

(51) Int. Cl. A61M 37/00

(52) U.S. Cl. 604/4.01

(57) ABSTRACT

An apparatus and a method is disclosed comprising one or more treatment chambers that may treat the blood of a patient in a minimally invasive way outside of the patient's body. Inside the chamber, the blood may be treated with heat, drugs, mechanical waves, electromagnetic waves, and material beams. In order to further minimize the side effect of treatments, the blood may first be separated into blood components. The treatments may then be applied only to those blood components that actually contain abnormal and cancerous cells that need to be killed.

### CROSS REFERENCE TO RELATED APPLICATION(S)

This present application claims the priority of a provisional application filed on Oct. 9, 2003, Ser. No. 60/509,586 titled “APPARATUS AND METHOD FOR TREATING BLOOD RELATED SICKNESSES” inventor and applicant Xiaoling Wang.

### FIELD OF THE INVENTION

This invention relates to the field of systems and methods for treating blood related illnesses.

### BACKGROUND OF THE INVENTION

Every year, many patients die of blood related illnesses, such as leukemia or multiple myeloma. Many of them die because their illnesses do not respond well to their treatments, such as chemotherapy. Some of them die because their bodies could not tolerate the severe side effects of their treatments. However, if we could treat the blood of a patient in a minimally invasive way outside of the patient's body in a treatment chamber, many of these patients' lives may be saved.

### SUMMARY OF THE INVENTION

The present invention, in one or more embodiments, introduces a system and a method that treats the blood of a patient in a minimally invasive way outside of the patient's body. The blood may first be drawn into a treatment chamber. Inside the chamber, the blood may be treated with a process. For example heat, or chemicals, such as drugs, may be applied to the blood. The blood may also or alternatively be treated with mechanical waves, such as ultrasound, or with electromagnetic waves, such as gamma rays, x-rays, ultraviolet rays, visible light, or infrared light. The blood may also or alternatively be treated with material beams, such as proton, electron, or neutron beams. The treatment options may be determined by physicians depending on the illness of a patient. In order to minimize the side effects of a treatment, the blood may also be separated into blood components, such as plasma, red cells, white cells and platelets. A treatment may then be applied only to those blood components that actually contain, for example, abnormal and cancerous cells that need to be killed. After a treatment, the treated or modified blood or the blood components may be returned to the body of the patient. Because the treatments are done in a treatment chamber outside of the patient's body, side effects on other body parts may also be reduced significantly.

The treatment chamber may use differentiable differences between normal and abnormal blood cells to damage abnormal blood cells in the blood while keeping normal cells in the blood. The treatment chamber may apply a first set of chemical agents to the blood which are designed to harm abnormal cells. The treatment chamber, or a subsequent treatment chamber, may thereafter apply a second set of chemical agents to the blood which are designed to neutralize the potential side effects of the first set of chemical agents.

The treatment chamber may be comprised of a plurality of sub-chambers. Each of the sub-chambers may subject blood to a different process.

### BRIEF DESCRIPTION OF THE DRAWINGS

FIG. 1 is a perspective view schematically illustrating the overall structure of one embodiment of the present invention;

FIG. 2 is a perspective view schematically illustrating the overall structure of another embodiment of the present invention with fluid control valves and a control device with a timer;

FIG. 3A is a sectional view schematically illustrating the overall structure of a heat treatment chamber with two sub-chambers;

FIG. 3B is a sectional view schematically illustrating the overall structure of a heat treatment chamber with two sub-chambers and fluid control valves and a control device with timer;

FIG. 4 is a sectional view schematically illustrating the overall structure of a radiation treatment chamber;

FIG. 5 is a sectional view schematically illustrating the overall structure of a drug treatment chamber; and

FIG. 6 is a perspective view schematically illustrating the overall structure of another embodiment of the present invention with a blood component separating device.

### DETAILED DESCRIPTION OF THE DRAWINGS

The present invention, in one or more embodiments, introduces a system, an apparatus, and a method that treats the blood of a patient in a minimally invasive way outside of the patient's body. Cancerous cells are known to be more sensitive to heat, chemotherapy, and radiation than normal cells. The main challenge in cancer treatment is how to kill cancerous cells while keeping side effects manageable. In order to keep side effects manageable, the treatments used should be able to do enough damage to cancerous cells but leave normal cells unharmed or at least less damaged. In general, when no other human body parts are involved in a treatment except the blood, fewer side effects may be expected. Therefore, if the blood of a patient can be drawn into a treatment chamber and treated in the chamber with heat or other methods such as radiation and chemotherapy, damage to other body parts may be reduced significantly.

Many treatments may be deployed after the blood of a patient is drawn into a treatment chamber. The blood in a treatment chamber may for example be treated in the following ways:
(1) Heat, generated using conventional methods, or using power ultrasound (see item 3) and infrared light (see item 4), since normal cells may tolerate higher temperatures than abnormal and cancerous cells;
(2) Chemicals, such as drugs for chemo therapy;
(3) Mechanical waves, such as power ultrasound for directly destroying abnormal and cancerous cells or to heating them up to a certain killing temperature;
(4) Electromagnetic waves, such as gamma rays, x-rays, ultraviolet rays, microwaves for killing abnormal and cancerous cells using radiation, or infrared light for heating abnormal and cancerous cells to a certain killing temperature; and
(5) Material beams, such as proton, electron, and neutron beams for killing abnormal and cancerous cells by radiation.

The above-mentioned treatment ways are not intended to be exhaustive. It may be feasible to combine several treatments for achieving a better treatment outcome.

Because the blood of a patient needs to be first drawn into a treatment chamber, and then treated within the treatment chamber, and finally returned back to the patient, the whole process is actually similar to a typical blood dialysis apparatus that cleans wastes from the blood. This waste cleaning job is normally done by the kidneys. However, if the kidneys of a patient fail, the blood must be cleaned artificially with a dialysis system. The blood travels through tubes to a dialyzer, which removes wastes and extra fluid. The cleaned blood then flows through another set of tubes back into the body. The main difference is that the apparatus according to the present invention uses one or more blood treatment chambers for killing abnormal and cancerous blood cells instead of a dialyzer for removing wastes in a dialysis apparatus. Because of that, in the present invention, all peripheral devices needed to draw blood from a patient, to monitor the patient's blood pressure, to monitor possible air bubbles in the blood, are ignored for clarity. Only treatment chambers will be described in detail.

A perspective view of a system, an apparatus, and a method according to one embodiment of the present invention is shown in FIG. 1. FIG. 1 shows an apparatus 100 comprised of a treatment chamber 120, an inflow device or pipe 140, and an outflow device or pipe 160. The inflow device 140 has an opening 140a and the outflow device 160 has an opening 160a. First blood can flow from a human body through opening 140a, through inflow device 140, and into chamber 120. The first blood may be changed or processed in treatment chamber 120 and may come out of chamber 120, as second blood, in a changed form, through outflow device 160 and through opening 160a, and may thereafter be supplied to the same human body.

The treatment chamber 120 may be comprised of sub-chambers depending on treatment options. The first blood may flow into the treatment chamber 120 and out of the treatment chamber 120 as second blood continuously with a given speed. The speed is set in such a way that the treatment of the first blood during the travel time of the first blood through the treatment chamber 120 is sufficiently effective.

When a treatment in the treatment chamber 120 needs a certain period of time to be effective, the blood of a patient, or first blood or first blood supply, may be drawn into the treatment chamber 120, through opening 140a and through inflow device 140, and then be kept in the treatment chamber 120 for a certain period of time. The certain period of time is selected in such a way that the treatment in the treatment chamber 120 may kill abnormal and cancerous cells in the first blood effectively while keeping normal cells unharmed or at least less damaged. Because the first blood must be kept in the treatment chamber 120 for a certain period of time, a control device with timer and fluid control valves may be needed in certain instances.

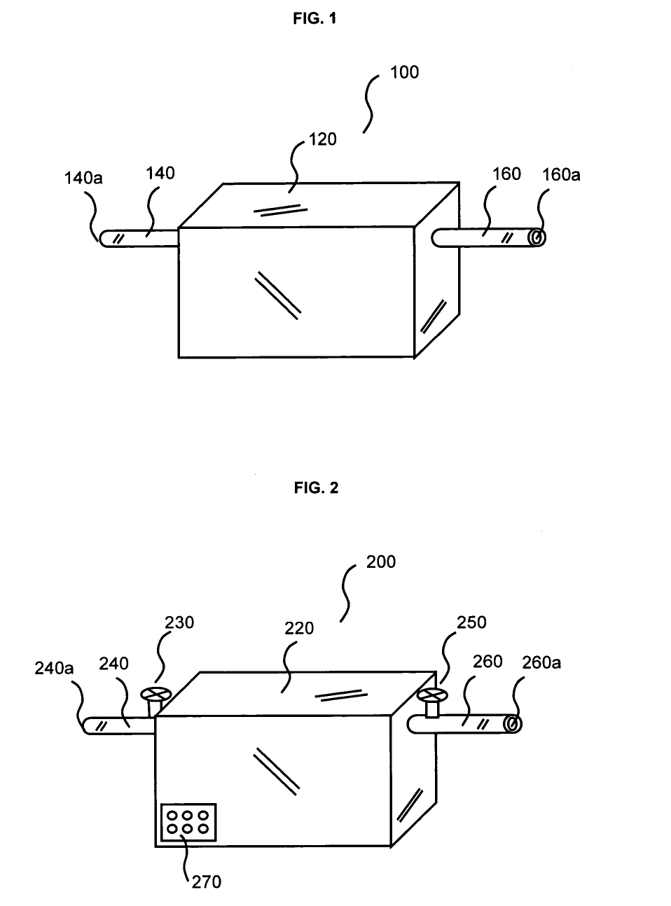

FIG. 2 depicts an apparatus 200 comprised of a treatment chamber 220, an inflow fluid control valve 230, an inflow device 240 having an opening 240a, an outflow fluid control valve 250, and an outflow device 260 having an opening 260a. First blood from a human being or patient may be received into chamber 220 through opening 240a and through inflow device 240, and through fluid control valve 230. The treatment chamber 220 may alter the first blood received to form the second blood, which may then be sent through outflow fluid control valve 250, through outflow device 260, and through opening 260a to the human being or patient.

The apparatus 200 and the chamber 220 also may include a control device with timer 270 for controlling when the inflow fluid control valve 230 and the outflow fluid control valve 250 should be opened. The treatment chamber 220 may be comprised of sub-chambers depending on treatment options.

Each of the treatment chambers 120 and 220, shown in FIG. 1 and FIG. 2 respectively, may have various external shapes and forms, such as for example a cylinder. Each of the treatment chambers 120 and 220 may also have various internal shapes and structures depending on the treatment options used for treating blood diseases. Several possible treatment chambers for typical treatments will be illustrated and discussed below.

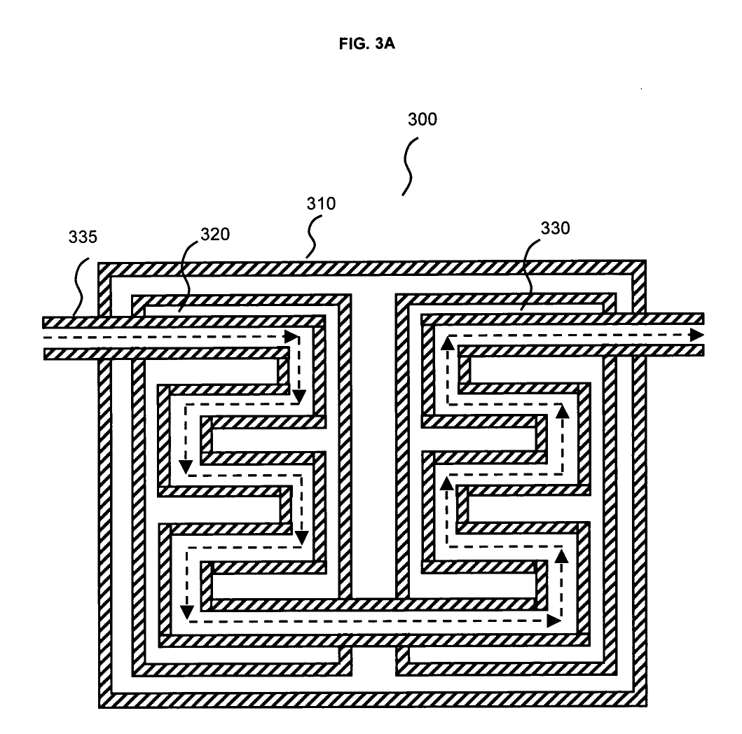

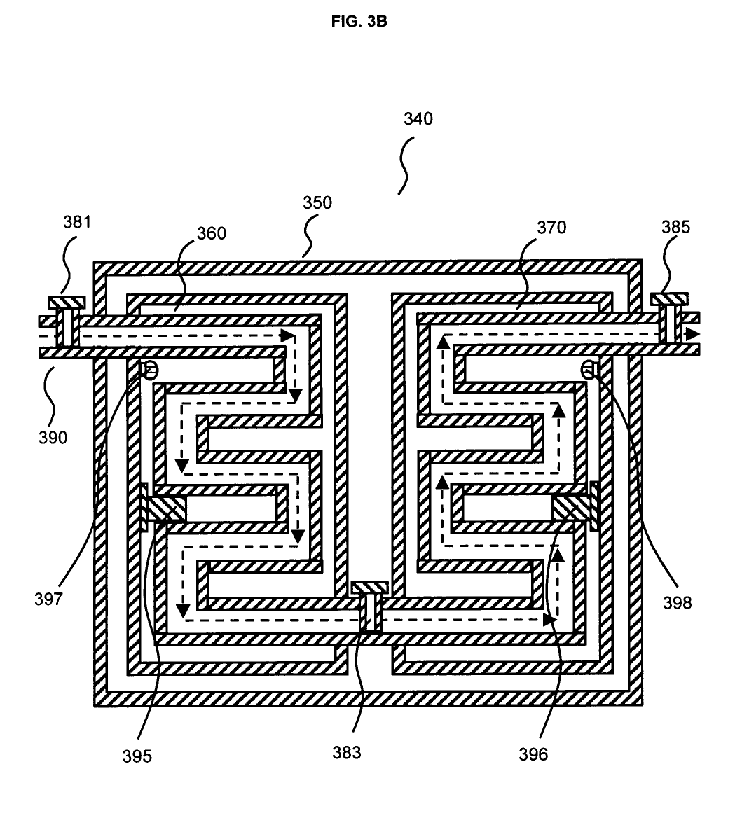

A sectional view of a treatment chamber 300 for heat treatment is shown in FIG. 3A. Treatment chamber 300 may be used as treatment chamber 120 or 220 in FIGS. 1 or 2, respectively. The treatment chamber 300 is comprised of an enclosure 310 with two separate sub-chambers 320 and 330. The first sub-chamber 320 has a first temperature T1, inside the first sub-chamber 320, while the second sub-chamber 330 has a second temperature T2, inside the second sub-chamber 330, where T1 and T2 are substantially different. In general, T1 is the heat treatment temperature with which abnormal and cancerous cells may be killed or damaged while normal cells may be unharmed or less damaged. In contrast, the temperature T2 is typically selected to be very close to a patient's (such as a human patient's) own blood temperature so that the discomfort caused by temperature differences between a patient's untreated blood and heat treated blood may be minimized when the treated blood flows back to the patient. For example, T1 may be one hundred eight or one hundred and twenty degrees Fahrenheit and T2 may be 98.6 degrees Fahrenheit. For different diseases, depending on treatment responses, T1 may vary in a certain range. The exact temperature T1 may be determined by a physician who treats a patient for optimum results. Therefore, the sub-chamber 330 may also be seen as a cooling down or a temperature normalization sub-chamber.

A pipe or tube 335 carries blood through both sub-chambers 320 and 330. Blood entering the treatment chamber 300, at the left of FIG. 3A, may be thought of as first blood, and blood exiting the chamber 300 at the right of FIG. 3A may be thought of as second blood, or modified blood. In the example of FIG. 3A, the pipe or tube 335 has multiple turns in both sub-chambers 320 and 330 for increasing heat exchange between the air in the sub-chambers 320 and 330 and the blood inside the pipe or tube 335. The blood flow inside the pipe 335 is shown by a dashed line with arrows showing the direction of blood flow.

In order to change the temperature of the blood quickly, it may be feasible to use fluid, such as water. For example, water may be provided at a temperature of T1 degrees in the sub-chamber 320, but outside of the pipe or tube 335. Water may also be provided at a temperature of T2 degrees in the sub-chamber 330, but outside of the pipe or tube 335. In general, fluid has higher heat capacity and can therefore heat up or cool down blood in the pipe or tube 335 faster than air. The water provided in sub-chamber 320 is typically separate from the water provided in sub-chamber 330. The water provided in sub-chamber 320 or in sub-chamber 330 contacts the outer surface of the pipe or tube 335 and should never be in direct contract with blood to avoid any possibility of contamination.

The heat treatment chamber 300, shown in FIG. 3A, may be extended to include fluid control valves. As shown in a sectional view in FIG. 3B, a heat treatment chamber 340 is comprised of an enclosure 350 with two separate sub-chambers 360 and 370, three fluid control valves 381, 383, and 385. The first sub-chamber 360 has a first temperature T3, inside the first sub-chamber 360, while the second sub-chamber 370 has a second temperature T4, inside the second sub-chamber 370, where T3 and T4 are substantially different. T3 and T4 used in the heat treatment chamber 340 may be selected to be identical or similar to the temperatures T1 and T2, respectively, used in the heat treatment chamber 300 shown in FIG. 3A. T3 is the heat treatment temperature with which abnormal and cancerous cells may be killed or damaged while normal cells may be unharmed or less damaged. The temperature T4 is selected to be very close to a patient's (such as a human patient's) own blood temperature so that the discomfort caused by temperature differences may be minimized when the treated blood flows back to the patient.

One of the main additional features of the heat treatment chamber 340 versus the heat treatment chamber 300 is the heat treatment chamber 340's capability to control how long the blood stays in the sub-chamber 360 for receiving the heat treatment and how long the blood stays in the sub-chamber 370 for normalizing the blood's temperature before returning blood, in a modified form, to the patient. The fluid control may be done using the three fluid control valves 381, 383, and 385. A pipe or tube 390 carries the blood through the treatment sub-chambers 360 and 370. The pipe or tube 390 may have multiple turns in both sub-chambers 360 and 370 for increasing heat exchange between the air or fluid inside the sub-chambers 360 and 370 and the blood inside the pipe or tube 390. In order to increase the speed and the homogeneity of the heat treatment, vibrating devices 395 and 396, similar to the vibration devices used in electronic vibration shavers but stronger, may be attached to the pipe 390, as shown in FIG. 3B. The vibrating devices 395 and 396 may be part of the sub-chambers 360 and 370, respectively. When the vibrating devices 395 and 396 are turned on, the pipe 390 vibrates with them so that the heat may distribute itself quickly and evenly in the blood. In addition to vibrating devices 395 and 396, it is also possible to built electric fans, such as electric fans 397 and 398, in each of the sub-chambers 360 and 370, respectively, to make the heat exchange between the sub-chambers 360 and 370 and the pipe or tube 390 better and faster.

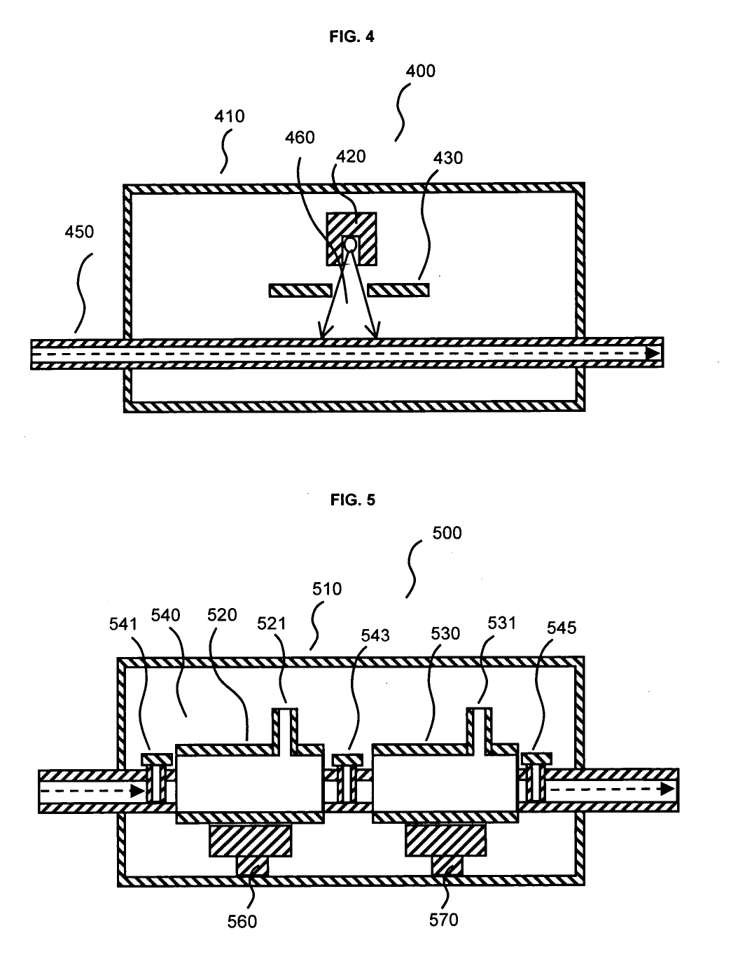

A sectional view of a treatment chamber 400 for radiation treatment is shown in FIG. 4. The treatment chamber 400 is comprised of an enclosure 410 that is capable of preventing leaking of radiation used in the treatment, a radiation source 420 for providing needed radiation, and a shutter 430 for controlling the dose and the timing of the radiation. The radiation source 420 may be a piece of radioactive material generating Gamma rays, an X-ray tube generating therapeutic X-rays, or an ultraviolet (UV) light source generating powerful ultraviolet rays. In addition to Gamma rays, X-rays and UV light, other electromagnetic waves, such as infrared (IR), and microwaves may also be used for killing or damaging abnormal and cancerous cells, as mentioned earlier. In general, different electromagnetic waves with the wavelength between 0.0001 and 0.01 nanometers (Gamma rays), 0.01 to 10 nanometers (X-ray), 10 nanometers to 420 nanometers (UV), 420 to 780 nanometers (Visible light), 780 nanometers to 0.1 millimeters (IR), 0.1 to 100 millimeters (Microwave), may be used to treat the blood. The radiation source 420 may even include sources capable of generating material beams, such as electron, neutron and proton beams. The shutter 430 is needed to control the radiation dose, as well as where and when and for how long the radiation is applied to the blood within enclosure 410. The pipe or tube 450 which carries blood through the enclosure 410 may expose the blood inside the pipe or tube 450 to the radiation near the area 460.

A sectional view of a treatment chamber 500 for drug treatment is shown in FIG. 5. The treatment chamber 500 is comprised of an enclosure 510 with two separate sub-chambers 520 and 530, a fluid control device 540 comprised of valves 541, 543, and 545. The first sub-chamber 520 has an opening 521 for receiving a first set of therapeutic drugs or chemical agents while the second sub-chamber 530 has also an opening 531 for receiving a second set of chemical agents for neutralizing the drugs used in the first sub-chamber 520. The fluid control device 540 comprised of valves 541, 543, and 545 determines how long the blood may stay in the first sub-chamber 520 for receiving treatment from the first set of drugs. The time may be selected in such a way that the first set of drugs has enough time to kill or substantially damage abnormal and cancerous cells in blood while keeping normal cells unharmed or less harmed. After that, the blood, or second blood, or blood in a modified form will be drawn into the second sub-chamber 530 where a second set of chemical agents may be added for neutralizing the first set of drugs. The neutralizing process should at least make the first set of chemical agents significantly less potent and less toxic. After a certain period of time, the neutralizing process is finished and the treated blood, or third blood, or further modified blood, may be returned to the patient. When the treated blood with the two sets of chemical agents flows back to a patient's body, the combined chemical agents with significantly reduced toxicity should cause significantly less side effects or even no side effects.

The main advantage of a drug treatment chamber with two sub-chambers is its ability to treat new blood in the first sub-chamber, such as sub-chamber 520 of FIG. 5, while the second sub-chamber, such as sub-chamber 530 of FIG. 5, is neutralizing the drugs given on the blood in a previous instance in the first sub-chamber, such as 520. When the neutralizing process is very fast, one sub-chamber may be sufficient. In the case of one treatment sub-chamber, the two sets of chemical agents may be added to the blood in the same treatment sub-chamber with a given time delay between them. For example, if the first set of drugs or chemical agents needs one hundred and ten seconds to kill or substantially damage abnormal and cancerous cells and the second set of chemical agents needs five seconds to neutralize the first set of drugs, then the whole treatment may take around two minutes. The first set of drugs will be added first to the blood. After one hundred and fifteen seconds, the second set of chemicals will be added. After another five seconds, the blood, in its modified form, is ready to be returned to the patient's body. In order to increase the speed and the homogeneity of the drug treatment, vibrating devices 560 and 570, shown in FIG. 5, may be used to vibrate the sub-chambers 520 and 530, respectively, so that the chemicals may mix with the blood quickly and evenly.

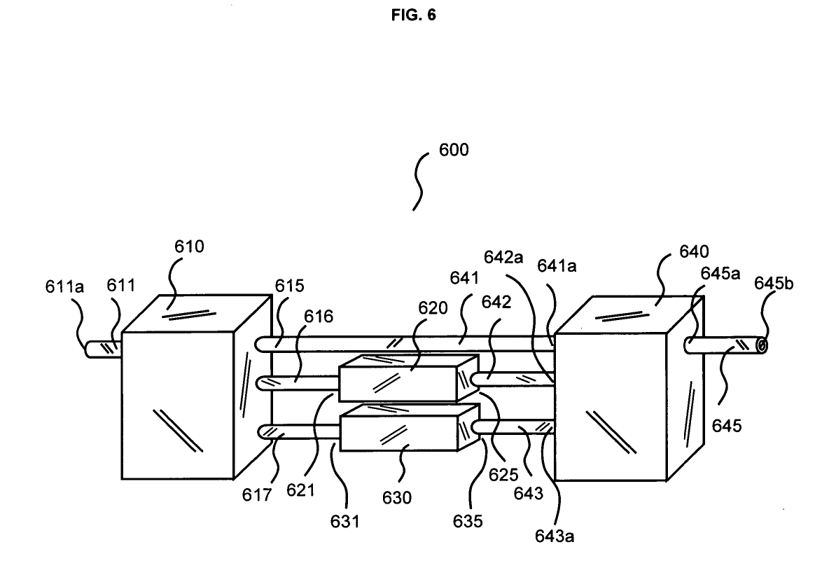

In order to further minimize the side effects of a treatment, the blood may first be separated into blood components, such as plasma, red cells, white cells and platelets. The blood components may be separated by centrifugation. After separating blood into blood components, treatments may be applied only to those blood components that actually contain, for example, abnormal and cancerous cells that need to be killed. This extra step may further reduce the side effects of a treatment. A perspective view of a system, an apparatus, and a method according to another embodiment of the present invention is shown in FIG. 6. FIG. 6 shows an apparatus 600 comprised of a blood component separating device 610, an inflow device or pipe 611, outflow devices or pipes 615, 616, and 617, for receiving different blood components. The inflow device 611 has an opening 611a for receiving blood from a patient such as a human patient.

The apparatus 600 is also comprised of a plurality of treatment chambers each having an inflow opening, such as treatment chambers 620 and 630 having inflow openings 621 and 631, respectively, for receiving different blood components. The treatment chambers 620 and 630 also include outflow openings 625 and 635 for feeding treated blood components from the treatment chambers 620 and 630, respectively to a blood component mixing device 640, via outflow devices or pipes 642 and 643. The blood component mixing device 640 may have multiple inflow openings, such as 641a, 642a and 643a, for receiving treated and untreated blood components from devices 641, 642, and 643, respectively. The device 640 may also have an outflow opening 645a leading to outflow device 645 and an outflow opening 645b for returning mixed treated blood to the patient.

The treatment chambers 620 and 630 may not be identical. In some embodiments one treatment method may be used for one blood component, and another treatment method may be used for another blood component. Even when the treatment chambers 620 and 630 are identical in working principle, the settings may also be different. For example, both treatment chambers 620 and 630 may be heat treatment chambers, like chamber 300, shown in FIG. 3A. However, the treatment heat temperature, may be different from T1 for FIG. 3A, and the cooling-off temperature may be different from T2 for FIG. 3A. Although the blood component separating device 610 may be able to separate blood into many different components, it should only separate blood into those components that require different treatments. For example, if red cells and white cells need different treatments, they need to be separated and fed into different treatment chambers, such as treatment chambers 620 and 630, respectively, for receiving different treatments. However, if red cells and platelets need an identical treatment, they should not be separated. In this case, both red cells and the platelets may be fed together into the treatment chamber 620 via outflow device or pipe 616. If the remaining blood components, such as plasma, do not need any treatment, they should by-pass the treatment chambers 620 and 630 and go directly into the mixing device 640 through device 641. Please note that the total number of the outflow devices or pipes, such as 615, 616, and 617 from the blood component separating device 610, as well as the total number of the treatment chambers, such as 620 and 630, shown in FIG. 6, may serve only as one example. Depending on real treatment needs, a system and method according to the present invention may use more or less blood component outflow devices, the total number of the treatment chambers, as well as the property of the treatment chambers.

Although the invention has been described by reference to particular illustrative embodiments thereof, many changes and modifications of the invention may become apparent to those skilled in the art without departing from the spirit and scope of the invention. For example, if a plurality of treatments is needed, then a plurality of treatment chambers may be daisy-chained together to form a treatment chain containing all needed treatments. In some other cases, one or more treatments may be combined and executed in one treatment chamber. For example, if both heat and radiation treatments are needed, it may be more cost-effective and time-saving to deliver radiation to the blood or the blood component while it is in the heat or the cooling-down sub-chamber. It is certainly possible to pack even more treatment methods into one treatment chamber. It is therefore intended to include within this patent all such changes and modifications as may reasonably and properly be included within the scope of the present invention's contribution to the art.

1. An apparatus comprising
a first treatment chamber; and
wherein first blood can flow through an inflow opening in the apparatus into the first treatment chamber; and
wherein the first treatment chamber uses differentiable differences between normal and abnormal blood cells to damage abnormal blood cells in the first blood while keeping normal cells in the first blood less damaged to form second blood.

2. The apparatus of claim 1 wherein
the first blood is supplied by a human body;
and the second blood is supplied to the human body through an outflow opening of the apparatus.

3. The apparatus of claim 1 wherein
the first blood has a first temperature; and
the first treatment chamber causes the second blood to have a temperature, which is substantially different from the first blood.

4. The apparatus of claim 2 wherein
the first blood and the second blood have substantially the same temperature.

5. The apparatus of claim 1 wherein
the first treatment chamber is comprised of an electromagnetic radiation device, which radiates the first blood in the first treatment chamber.

6. The apparatus of claim 5 wherein
the electromagnetic radiation device radiates the first blood with Gamma rays.

7. The apparatus of claim 5 wherein
the electromagnetic radiation device radiates the first blood with X rays.

8. The apparatus of claim 5 wherein
the electromagnetic radiation device radiates the first blood with ultraviolet rays.

9. The apparatus of claim 5 wherein
the electromagnetic radiation device radiates the first blood with infrared energy.

10. The apparatus of claim 5 wherein
the electromagnetic radiation device radiates the first blood with microwaves.

11. The apparatus of claim 5 wherein
the electromagnetic radiation device radiates the first blood with electromagnetic waves in a wavelength range between 0.0001 nanometer and 100 millimeters

12. The apparatus of claim 1 wherein
the first treatment chamber is comprised of a mechanical wave radiation device, which radiates the first blood in the first treatment chamber.

13. The apparatus of claim 12 wherein
the mechanical wave radiation device radiates the first blood with ultrasonic waves.

14. The apparatus of claim 1 wherein
the first treatment chamber is comprised of a drug treatment device, which treats the first blood in the first treatment chamber with chemical agents.

15. The apparatus of claim 14 wherein
the drug treatment device treats the first blood with two sets of chemical agents;
wherein the first set of chemical agents is designed to harm abnormal cells in the first blood; and
wherein the second set of chemical agents is designed to neutralize the first set of chemical agents in order to minimize side effects associated with the first set of chemical agents when the second blood is supplied to a human body.

16. The apparatus of claim 1 wherein
the first treatment chamber is comprised of a material beam radiation device, which radiates the first blood in the first treatment chamber.

17. The apparatus of claim 16 wherein
the material beam radiation device uses protons to radiate the first blood.

18. The apparatus of claim 16 wherein
the material beam radiation device uses electrons to radiate the first blood.

19. The apparatus of claim 16 wherein
the material beam radiation device uses neutrons to radiate the first blood.

20. The apparatus of claim 1 wherein
the first treatment chamber is comprised of a plurality of sub-chambers.

21. The apparatus of claim 20 wherein
the first treatment chamber is comprised of a first sub-chamber and a second sub-chamber;
wherein the first sub-chamber is connected to the second sub-chamber;
wherein the first sub-chamber partially modifies the first blood to form first partially modified blood by subjecting the first blood to a first process;
wherein the second sub-chamber partially modifies the first partially modified blood to form the second blood by subjecting the first partially modified blood to a second process;
wherein the first and the second processes are different.

22. The apparatus of claim 21 wherein
the second sub-chamber is comprised of a plurality of further chambers.

23. The apparatus of claim 21 wherein
the second sub-chamber changes the temperature of the first partially modified blood to form the second blood.

24. The apparatus of claim 21 wherein
the second sub-treatment chamber is comprised of an electromagnetic radiation device, which radiates the first partially modified blood to form the second blood.

25. The apparatus of claim 21 wherein
the second sub-treatment chamber is comprised of a mechanical wave radiation device, which radiates the first partially modified blood to form the second blood.

26. The apparatus of claim 21 wherein
the second sub-treatment chamber is comprised of a drug treatment device, which treats the first partially modified blood with chemical agents to form the second blood.

27. The apparatus of claim 21 wherein
the second sub-treatment chamber is comprised of a material beam radiation device, which radiates the first partially modified blood to form the second blood.

28. An apparatus comprising
an inflow device;
an outflow device;
a treatment chamber; and
wherein first blood from a human body can flow via the inflow device into the treatment chamber; and
wherein the first blood is subjected to a process in the treatment chamber to form second blood; and
wherein the second blood can flow from the treatment chamber via the outflow device to the human body.

29. The apparatus of claim 28 wherein
the treatment chamber includes an electromagnetic wave device and the process includes using the electromagnetic wave device to radiate the first blood with electromagnetic waves.

30. The apparatus of claim 29 wherein
the electromagnetic wave device radiates the first blood with electromagnetic waves in the wavelength range between 0.0001 nanometers and 100 millimeters.

31. The apparatus of claim 28 wherein
the treatment chamber includes a mechanical wave device and the process includes using the mechanical wave device to apply mechanical waves to the first blood.

32. The apparatus of claim 31 wherein
the mechanical wave device applies ultrasonic waves to the first blood to form the second blood.

33. The apparatus of claim 28 wherein
the treatment chamber includes a material beam device and the process includes applying material beams to the first blood.

34. An apparatus comprising
an inflow device;
an outflow device;
first and second treatment chambers; and
wherein first blood from a human body can flow via the inflow device into the first treatment chamber; and
wherein the first and second treatment chambers are connected; and
wherein the blood can flow into the first treatment chamber subjects blood supplied by the inflow device to a first process to form second blood;
wherein the second blood can flow into the second treatment chamber and the second treatment chamber subjects the second blood to a second process to form third blood; and
wherein the third blood flows from the second treatment chamber and at least part of the third blood can flow via the outflow device to the human body;
wherein the first and second processes are substantially different.

35. An apparatus comprising
an inflow device;
an outflow device;
a blood separation device;
a first treatment chamber;
a blood component mixing device; and
wherein first blood from a human body can flow via the inflow device into the blood separation device; and
wherein the blood separation device separates first blood into a plurality of blood components; and
wherein at least one of the plurality of blood components is sent to the first treatment chamber and therein subjected to a first treatment;
wherein the plurality of blood components are mixed in the blood component mixing device to form mixed blood;
wherein the mixed blood further can flow from the blood component mixing device via the outflow device back to the human body.

36. The apparatus of claim 35 further comprising
a second treatment chamber;
wherein at least one of the plurality of blood components is sent to the second treatment chamber and therein subjected to a second treatment;
wherein the second treatment and the first treatment are substantially different.

37. A method comprising the steps of
allowing first blood to flow into a treatment chamber;
wherein the first blood included normal and abnormal blood cells;
and further comprising treating the first blood in the treatment chamber to form second blood flowing out of the treatment chamber;
wherein the first blood is treated in the treatment chamber by using differentiable differences between the normal and abnormal blood cells in the first blood to damage the abnormal cells in the first blood while keeping the normal cells in the first blood less damaged.

38. The method of claim 37 wherein
the first blood is supplied by a human body; and the second blood is supplied to the human body.

39. The method of claim 37 wherein
the first blood is treated by subjecting the first blood to a temperature change; and
the differentiable differences between the normal and abnormal blood cells of the first blood which are used include differentiable temperature tolerance differences between normal and abnormal cells of the first blood.

40. The method of claim 37 wherein
the first blood is treated by subjecting the first blood to electromagnetic radiation; and
the differentiable differences between the normal and abnormal blood cells of the first blood which are used include differentiable tolerance to the electromagnetic radiation.

41. The method of claim 37 wherein
the first blood is treated by subjecting the first blood to material beam radiation;
and wherein the differentiable differences between the normal and abnormal blood cells of the first blood which are used include differentiable tolerance to the material beam radiation.

42. The method of claim 37 wherein
the first blood is treated by subjecting the first blood to mechanical wave radiation;
and wherein the differentiable differences between the normal and abnormal blood cells of the first blood which are used include differentiable tolerance to the mechanical wave radiation.

43. The method of claim 37 wherein
the first blood is treated by applying a drug to the first blood;
and wherein the differentiable differences between the normal and abnormal blood cells of the first blood which are used include differentiable tolerance to the drug.

44. A method comprising the steps of
separating a first blood into a plurality of blood components;
wherein at least a first blood component of the plurality of blood components is comprised of normal and abnormal cells; and
further comprising allowing the first blood component flow into a treatment chamber; treating the first blood component in the treatment chamber by using differentiable differences between the normal and abnormal blood cells of the first blood component to damage the abnormal blood cells while keeping normal cells less damaged to form a treated first blood component at the output of the treatment chamber.

45. The method of claim 44 further comprising
mixing the treated first blood component and one or more of the other blood components of the plurality of blood components to form a second blood.

46. The method of claim 45 wherein
the first blood is supplied by a human body;
and the second blood is supplied to the human body.

47. The method of claim 44 wherein
the first blood component is treated by subjecting the first blood component to a temperature change; and
the differentiable differences between the normal and abnormal blood cells of the first blood component which are used include differentiable temperature tolerance differences between normal and abnormal cells of the first blood component.

48. The method of claim 44 wherein
the first blood component is treated by subjecting the first blood component to electromagnetic radiation; and
the differentiable differences between the normal and abnormal blood cells of the first blood component which are used include differentiable tolerance to the electromagnetic radiation.

49. The method of claim 44 wherein
the first blood component is treated by subjecting the first blood component to material beam radiation;
and wherein the differentiable differences between the normal and abnormal blood cells of the first blood component which are used include differentiable tolerance to the material beam radiation.

50. The method of claim 44 wherein
the first blood component is treated by subjecting the first blood component to mechanical wave radiation;
and wherein the differentiable differences between the normal and abnormal blood cells of the first blood component which are used include differentiable tolerance to the mechanical wave radiation.

51. The method of claim 44 wherein
the first blood component is treated by applying a drug to the first blood component;
and wherein the differentiable differences between the normal and abnormal blood cells of the first blood component which are used include differentiable tolerance to the drug.
